(19) United States
(12) Patent Application Publication
(10) Pub. No.: US 2021/0145941 A1
(43) Pub. Date: May 20, 2021

(54) PHARMACEUTICAL COMPOSITIONS FOR TREATING ACID SPHINGOMYELINASE DEFICIENCY

(71) Applicant: Genzyme Corporation, Cambridge, MA (US)

(72) Inventors: Mark Yang, Wellesley, MA (US); Claudia Buser, Sudbury, MA (US); Bernardo Perez-Ramirez, Brookline, MA (US); Grant Trierweiler, Framingham, MA (US); Sangeeta Benjwal, Newton, MA (US)

(73) Assignee: Genzyme Corporation, Cambridge, MA (US)

(21) Appl. No.: 17/104,593

(22) Filed: Nov. 25, 2020

Related U.S. Application Data
(63) Continuation of application No. PCT/US2019/033983, filed on May 24, 2019.
(60) Provisional application No. 62/676,525, filed on May 25, 2018.

(51) Int. Cl. A61K 38/46; A61K 9/16; A61K 9/19; A61K 9/08

(52) U.S. Cl. CPC A61K 38/465; A61K 9/1623; A61K 9/08; A61K 9/1617; A61K 9/19; A61K 9/1611

(57) ABSTRACT

The present invention provides compositions such as aqueous liquid compositions and lyophilized compositions comprising a recombinant human acid sphingomyelinase. Provided also are methods for using the compositions to treat patients who are deficient in acid sphingomyelinase.

### CROSS REFERENCE TO RELATED APPLICATION

This application is a continuation of PCT/US2019/033983, filed on May 24, 2019, which claims priority from U.S. Provisional Application 62/676,525, filed May 25, 2018. The contents of the aforementioned priority applications are incorporated by reference herein in their entirety.

### SEQUENCE LISTING

The instant application contains a Sequence Listing which has been submitted electronically in ASCII format and is hereby incorporated by reference in its entirety. Said ASCII copy, created on Nov. 24, 2020, is named 022548_US048_SL.txt and is 31,325 bytes in size.

### BACKGROUND OF THE INVENTION

Acid sphingomyelinase deficiency (ASMD) is a rare life-threatening lysosomal storage disorder. It is an autosomal recessive genetic disease that results from mutations in the SMPD1 gene encoding the lysosomal enzyme acid sphingomyelinase (ASM) (Schuchman et al., Mol. Genet. Metab. 120(1-2):27-33 (2017)). ASMD patients are unable to metabolize sphingomyelin, which as a result accumulates in lysosomes in multiple organs, causing visceral disease and neurodegeneration in severe cases. ASMD patients have increased cholesterol and other lipids in spleen, liver, lung and bone marrow.

Infantile neurovisceral ASMD (also known as Niemann-Pick disease type A or NPD A) is the most severe disease phenotype and is characterized as the early-onset and acute neuropathic form. NPD A results in failure to thrive, hepatosplenomegaly, and rapidly progressive neurodegeneration. Patients die in early childhood (McGovern et al., Neurology 66(2):228-232 (2006)).

Patients with chronic visceral ASMD (NPD B) and chronic neurovisceral ASMD (NPD A/B) have onset that varies from infancy to adulthood (Wasserstein et al., Pediatrics 114(6):e672-677 (2004); Wasserstein et al., J. Pediatr. 149(4):554-559 (2006)). NPD B patients are usually diagnosed in childhood, typically after the age of two years. Most NPD B patients live to adulthood. NPD A/B patients are classified as having an intermediate form, with manifestation of childhood neurologic symptoms that may develop as neurodegenerative disease. Morbidity from liver, lung, and hematologic disease occurs in all patients with chronic ASMD and includes hepatosplenomegaly, liver dysfunction, infiltrative lung disease, and thrombocytopenia (McGovern et al., Genet. Med. 15(8):618-623 (2013); McGovern et al., Orphanet J. Rare Dis. 12(1):41 (2017)). Growth restriction during childhood and bone disorders such as low bone density are also common features of chronic ASMD (Wasserstein et al., J. Pediatr. 142(4):424-428 (2003)). Pulmonary and liver diseases are the main causes of death in these patients (McGovern et al., Pediatrics 122(2):e341-349 (2008); Cassiman et al., Mol. Genet. Metab. 118(3):206-213 (2016)).

Due to the high morbidity and mortality rates of ASMD, there remains an urgent need for an effective treatment of this genetic disease.

### SUMMARY OF THE INVENTION

The present invention provides compositions of recombinant human ASM (rhASM) for treating ASMD. In some embodiments, the compositions comprise rhASM, sodium phosphate, methionine, and sucrose (or trehalose). In certain embodiments, the rhASM is olipudase alfa (SEQ ID NO:2).

In some embodiments, the composition is lyophilized. A lyophilized composition of the invention may comprise, for example:

4-7% w/w olipudase alfa,

3-7% w/w sodium phosphate,

15-25% w/w L-methionine, and

65-75% w/w sucrose.

In certain embodiments, a lyophilized composition of the invention may comprise:

5.5% w/w olipudase alfa,

2.3% w/w sodium phosphate dibasic heptahydrate,

2.6% w/w sodium phosphate monobasic monohydrate,

20.5% w/w L-methionine, and

68.6% w/w sucrose.

In some embodiments, the composition is an aqueous liquid composition. The aqueous liquid composition may comprise, for example:

1-10 mg/mL olipudase alfa,

10-50 mM sodium phosphate,

70-150 mM L-methionine, and

1-10% w/v sucrose,

wherein the composition has a pH of 5-8.
In certain embodiments, an aqueous liquid composition of the invention may comprise:

3-5 mg/mL olipudase alfa,

10-30 mM sodium phosphate,

80-120 mM L-methionine, and

4-6% w/v sucrose,

wherein the composition has a pH of 6-7.

In a particular embodiment, an aqueous liquid composition of the invention may comprise:

4 mg/mL olipudase alfa,

20 mM sodium phosphate,

100 mM L-methionine, and

5% w/v sucrose,

wherein the composition has a pH of 6.5.

In some embodiments, the aqueous liquid composition of the invention may further comprise 0.005% w/v polysorbate 80.

The invention further provides a composition obtained by drying (e.g., lyophilizing or spray-drying) an aqueous liquid composition described herein. The invention also provides a process for manufacturing a lyophilized composition, comprising lyophilizing an aqueous liquid composition described herein.

In some embodiments, the invention provides a vial containing a lyophilized composition described herein. In certain embodiments, the lyophilized composition in the vial comprises, or consists essentially of:

21.2 mg olipudase alfa,

9.0 mg sodium phosphate dibasic heptahydrate,

10.0 mg sodium phosphate monobasic monohydrate,

79 mg L-methionine, and

265 mg sucrose.

In some embodiments, the lyophilized composition is reconstituted in 5.1 mL of sterile water to obtain an aqueous liquid composition.

In certain embodiments, the lyophilized composition in the vial comprises, or consists essentially of:

4.8 mg olipudase alfa,

2.0 mg sodium phosphate dibasic heptahydrate,

2.3 mg sodium phosphate monobasic monohydrate,

17.9 mg L-methionine, and

60 mg sucrose.

In some embodiments, the lyophilized composition is reconstituted in 1.1 mL of sterile water to obtain an aqueous liquid composition

The invention further provides an article of manufacture comprising 1) a vial containing a lyophilized composition described herein, and 2) a vial containing, e.g., sterile water, 0.9% sodium chloride, or phosphate-buffered saline for reconstituting the lyophilized composition.

The invention further provides a method of treating ASMD in a human patient, comprising administering to the patient a composition described herein, wherein the composition is reconstituted into a liquid form prior to administration if it is a lyophilized composition.

The invention further provides a composition described herein for use in treating ASMD in a human patient.

The invention further provides use of a composition described herein for the manufacture of a medicament for treating ASMD in a human patient.

In some embodiments, treatment of ASMD as described herein is for Niemann-Pick Disease type AB or type B or for non-neurological manifestations of ASMD.

### BRIEF DESCRIPTION OF THE FIGURES

FIG. 1A shows stability of rhASM as measured by specific (enzymatic) activity after two weeks of storage at 30° C. in a succinate, citrate, citrate/phosphate, or phosphate buffer at various pHs.

FIG. 1B shows the stability of rhASM as measured by the percent of high molecular weight species (% HMWS) after one week of storage at 30° C. in a succinate, citrate, citrate/phosphate, or phosphate buffer at various pHs. HMWS were determined by size exclusion chromatography (SEC).

FIG. 1C shows the stability of rhASM as measured by thermal stability in a citrate/phosphate or phosphate buffer at various pHs. Thermal stability was determined by differential scanning calorimetry.

FIG. 2A shows the specific activity of rhASM over time in a 10 mM, 20 mM, 50 mM, or 100 mM phosphate buffer with a pH of 6.5 at 30° C.

FIG. 2B shows the physical stability of rhASM over time as measured by % HMWS in a 10 mM, 20 mM, 50 mM, or 100 mM phosphate buffer with a pH of 6.5 at 30° C.

FIG. 3A shows the effects of 5% w/v mannitol, sucrose, or trehalose on the specific (enzymatic) activity of 4 mg/mL rhASM before lyophilization (liquid) and after lyophilization (lyo).

FIG. 3B shows the effects of 5% w/v mannitol, sucrose, and trehalose on the physical stability of 4 mg/mL rhASM as measured by % HMWS before lyophilization (liquid) and after lyophilization (lyo).

FIG. 4A shows the specific activity of rhASM over time at 5° C. The rhASM was lyophilized from a solution containing 5% mannitol, 5% sucrose, or 3% mannitol and 2% sucrose (all w/v concentrations).

FIG. 4B shows the physical stability of rhASM over time at 5° C. as measured by % HMWS. The rhASM was lyophilized from a solution containing 5% mannitol, 5% sucrose, or 3% mannitol and 2% sucrose (all w/v concentrations).

FIG. 5A shows the specific activity of rhASM over time at 5° C. The rhASM was lyophilized from a solution containing 5% sucrose with or without 100 mM methionine (all w/v concentrations).

FIG. 5B shows the physical stability of rhASM over time at 5° C. as measured by % HMWS. The rhASM was lyophilized from a solution containing 5% w/v sucrose with or without 100 mM methionine.

FIG. 6 shows the effects of pH, protein concentration, methionine concentration, and sucrose concentration on the percentage of dimer over time in liquid rhASM compositions at 2-8° C.

FIG. 7 shows the effects of pH, protein concentration, methionine concentration, and sucrose concentration on rhASM specific activity over time in liquid compositions at 2-8° C.

FIG. 8 shows the % HMWS over time at 2-8° C. in liquid rhASM compositions with varying pH, protein concentration, methionine concentration, and sucrose concentration. Formulation numbers are indicated to the right of the graph.

FIG. 9 shows the % aggregation over time at 2-8° C. in liquid rhASM compositions with varying pH, protein concentration, methionine concentration, and sucrose concentration. Formulation numbers are indicated to the right of the graph.

FIG. 10 shows the % dimer, % aggregation, and specific activity at 25° C. in liquid rhASM compositions at various pHs.

### DETAILED DESCRIPTION OF THE INVENTION

The present invention provides compositions comprising a recombinant human ASM, e.g., olipudase alfa, and one or more pharmaceutically acceptable excipients. The compositions of the present disclosure have improved stability and shelf life as compared to other compositions. In some embodiments, the compositions of the invention are pharmaceutical compositions, i.e., compositions that are in such a form, or can be prepared to become such a form, as to permit the biological activity of the active ingredient to be effective while containing no additional ingredients that are significantly toxic or otherwise cause unwanted side effects not related to the active ingredient in patients. The terms “pharmaceutical composition” and “pharmaceutical preparation” are used interchangeably herein. The pharmaceutical compositions of the present invention are useful in treating patients with ASM deficiency as further described below.

### Recombinant Human Acid Sphingomyelinase

ASM is an enzyme catalyzing the breakdown of sphingomyelin to ceramide and phosphorylcholine. “Recombinant human ASM” refers to human ASM, with or without certain amino acid modifications relative to a wildtype sequence, that is prepared by recombinant means. For example, a recombinant human ASM may be expressed in cultured mammalian host cells (e.g., COS, CHO, HeLa, 3T3, 293T, NS0, SP2/0, or HuT 78 cells and the like) or in animals transgenic for a human ASM coding sequence.

In some embodiments, the recombinant human ASM is olipudase alfa. Olipudase alfa is the glycoform alpha of a human ASM (EC-3.1.4.12) produced in CHO cells. Mature olipudase alfa is a 570 amino acid polypeptide that retains the enzymatic and lysosomal targeting activity of the native human protein. The amino acid sequence of olipudase alfa, including its leader sequence (residues 1-57), is shown below as SEQ ID NO:1, where the leader sequence is italicized and in boldface. The mature olipudase alfa sequence (SEQ ID NO:2, which spans residues 58-627 of SEQ ID NO:1) does not have the leader sequence.

(SEQ ID NO: 1)

[symbol]

[symbol]

HPL SPQGHPARLH RIVPRLRDVF GWGNLTCPIC KGLFTAINLG
LKKEPNVARV GSVAIKLCNL LKIAPPAVCQ SIVHLFEDDM VEVWRRSVLS
PSEACGLLLG STCGHWDIFS SWNISLPTVP KPPPKPPSPP APGAPVSRIL
FLTDLHWDHD YLEGTDPDCA DPLCCRRGSG LPPASRPGAG YWGEYSKCDL
PLRTLESLLS GLGPAGPFDM VYWTGDIPAH DVWHQTRQDQ LRALTTVTAL
VRKFLGPVPV YPAVGNHEST PVNSFPPPFI EGNHSSRWLY EAMAKAWEPW
LPAEALRTLR IGGFYALSPY PGLRLISLNM NFCSRENFWL LINSTDPAGQ
LQWLVGELQA AEDRGDKVHI IGHIPPGHCL KSWSWNYYRI VARYENTLAA
QFFGHTHVDE FEVFYDEETL SRPLAVAFLA PSATTYIGLN PGYRVYQIDG
NYSGSSHVVL DHETYILNLT QANIPGAIPH WQLLYRARET YGLPNTLPTA
WHNLVYRMRG DMQLFQTFWF LYHKGHPPSE PCGTPCRLAT LCAQLSARAD
SPALCRHLMP DGSLPEAQSL WPRPLFC

In other embodiments, the human ASM useful in the present invention is 99%, 98%, 97%, 96%, or 95% identical in amino acid sequence to olipudase alfa. For example, the human ASM in the composition may have the sequence shown in U.S. Pat. No. 6,541,218, the disclosure of which is incorporated herein in its entirety. That sequence (SEQ ID NO:3) is shown below, with the leader sequence (residues 1-59) italicized and in boldface, where the mature protein (SEQ ID NO:4, which spans residues 60-629 of SEQ ID NO:3) does not have the leader sequence.

(SEQ ID NO: 3)

[symbol]

[symbol]

H PLSPQGHPAR LHRIVPRLRD VFGWGNLTCP ICKGLFTAIN
LGLKKEPNVA RVGSVAIKLC NLLKIAPPAV CQSIVHLFED DMVEVWRRSV
LSPSEACGLL LGSTCGHWDI FSSWNISLPT VPKPPPKPPS PPAPGAPVSR
ILFLTDLHWD HDYLEGTDPD CADPLCCRRG SGLPPASRPG AGYWGEYSKC
DLPLRTLESL LSGLGPAGPF DMVYWTGDIP AHDVWHQTRQ DQLRALTTVT
ALVRKFLGPV PVYPAVGNHE SIPVNSFPPP FIEGNHSSRW LYEAMAKAWE
PWLPAEALRT LRIGGFYALS PYPGLRLISL NMNFCSRENF WLLINSTDPA
GQLQWLVGEL QAAEDRGDKV HIIGHIPPGH CLKSWSWNYY RIVARYENTL
AAQFFGHTHV DEFEVFYDEE TLSRPLAVAF LAPSATTYIG LNPGYRVYQI
DGNYSRSSHV VLDHETYILN LTQANIPGAI PHWQLLYRAR ETYGLPNTLP
TAWHNLVYRM RGDMQLFQTF WFLYHKGHPP SEPCGTPCRL ATLCAQLSAR
ADSPALCRHL MPDGSLPEAQ SLWPRPLFC

The human ASM in the composition may also be identical in amino acid sequence to the human ASM disclosed in the UNIPROT database as sequence P17405-1, or polymorphic variants thereof. The P17405-1 sequence is shown below (SEQ ID NO:5), with the leader sequence (residues 1-59) italicized and in boldface, where the mature protein (SEQ ID NO:6, which spans residues 60-629 of SEQ ID NO:5) does not have the leader sequence.

(SEQ ID NO: 5)

[symbol]

[symbol]

H PLSPQGHPAR LHRIVPRLRD VFGWGNLTCP ICKGLFTAIN
LGLKKEPNVA RVGSVAIKLC NLLKIAPPAV CQSIVHLFED DMVEVWRRSV
LSPSEACGLL LGSTCGHWDI FSSWNISLPT VPKPPPKPPS PPAPGAPVSR
ILFLTDLHWD HDYLEGTDPD CADPLCCRRG SGLPPASRPG AGYWGEYSKC
DLPLRTLESL LSGLGPAGPF DMVYWTGDIP AHDVWHQTRQ DQLRALTTVT
ALVRKFLGPV PVYPAVGNHE STPVNSFPPP FIEGNHSSRW LYEAMAKAWE
PWLPAEALRT LRIGGFYALS PYPGLRLISL NMNFCSRENF WLLINSTDPA
GQLQWLVGEL QAAEDRGDKV HIIGHIPPGH CLKSWSWNYY RIVARYENTL
AAQFFGHTHV DEFEVFYDEE TLSRPLAVAF LAPSATTYIG LNPGYRVYQI
DGNYSGSSHV VLDHETYILN LTQANIPGAI PHWQLLYRAR ETYGLPNTLP
TAWHNLVYRM RGDMQLFQTF WFLYHKGHPP SEPCGTPCRL ATLCAQLSAR
ADSPALCRHL MPDGSLPEAQ SLWPRPLFC

### Recombinant Human Acid Sphingomyelinase Compositions

The compositions of the present invention contain a recombinant human ASM and demonstrate superior stability with respect to the enzyme. “Stable” or “stability” refers to the ability of an active ingredient in a composition to retain its physical stability, chemical stability, and/or biological activity during storage, and/or when subjected to physical or chemical stress. Stability can be in the context of a selected temperature, for example, under refrigerated conditions (e.g., 2-8° C.), or at room temperature (e.g., 23-25° C.), for a selected time period, e.g., 16 weeks, 24 weeks, 36 weeks, four months, six months, one year, two years, three years, or longer. Stability of a protein may be measured in assays that are conducted within a shorter period of time but whose results are indicative of stability in clinical settings. Such assays include freeze/thaw assays where a protein composition is subjected to one or more freeze-thaw cycles; or agitation assays where a protein composition is subjected to mechanic agitation treatment over a pre-determined period. Protein stability may be determined by storing the protein composition at a designated storage temperature (such as 2-8° C.) over a selected time period and analyzing its structural and functional attributes, such as degree of dimerization or aggregation (e.g., as measured by size exclusion HPLC or protein gel), protein degradation (e.g., as measured by size exclusion HPLC or protein gel), color change of the composition, clarity of a liquid composition, enzymatic activity, glycan content and composition, receptor binding affinity, methionine residual oxidation, and the biological activity of the composition.

The compositions of the present invention contain one or more pharmaceutically acceptable excipients. “Excipient” refers to an inert substance that is used as a diluent, vehicle, carrier, preservative, binder, or stabilizing agent for the active ingredient(s) of a drug. For example, the compositions may contain a buffering agent, an isotonic agent, and/or a stabilizing agent such as an anti-oxidant. In some cases, one agent may serve more than one of these purposes. In some embodiments, a composition of the invention contains a recombinant human ASM such as olipudase alfa, a buffering agent such as sodium phosphate or sodium citrate, a stabilizer such as L-methionine, and a nonreducing sugar such as sucrose or trehalose. The human ASM has improved stability due to the particular makeup in the composition. The compositions of the invention may be aqueous liquid solutions or lyophilized preparations.

Liquid Compositions

In some embodiments, the composition is an aqueous liquid composition comprising 1-10 mg/mL (e.g., 3-5 mg/mL) rhASM (e.g., olipudase alfa); 10-50 mM (e.g., 10-30 mM) sodium phosphate; 70-150 mM (e.g., 80-120 mM) methionine (e.g., L-methionine); and 1-10% (e.g., 4-6%) w/v sucrose or trehalose. The pH of the aqueous liquid composition may be 5-8 (e.g., 6-7).

In some embodiments, the aqueous liquid composition comprises no detectable amount of mannitol, the most readily used crystalline excipient, because it may significantly increase aggregation of the human ASM during or after the lyophilization of an aqueous liquid composition described herein.

In some embodiments, the aqueous liquid composition comprises 0.004-0.008%, 0.005-0.007%, or 0.005% w/v surfactant(s). Exemplary surfactants include nonionic detergents, such as polysorbates (e.g., polysorbates 20 and 80) and poloxamers (e.g., poloxamer 188). In a particular embodiment, the aqueous liquid composition comprises 0.005% polysorbate 80. In some cases, the presence of surfactant(s) may help to reduce turbidity in the liquid composition.

In some embodiments, the aqueous liquid composition comprises no more than 0.05, 0.01, or 0.005 mM chelating agent(s), such as EDTA and EGTA; in an exemplary embodiment, the aqueous liquid composition comprises no detectable amount of chelating agent(s). In some cases, the presence of chelating agents at a concentration above, e.g., 0.05 mM or 0.1 mM may increase aggregation of the human ASM and decrease its stability, particularly after a prolonged storage period, e.g., for 12-16 weeks, or under non-refrigerated conditions, e.g., at 25° C.

In some embodiments, the aqueous liquid composition may contain 0-50 ppm (e.g. 15-30 ppm) of zinc, which may be, e.g., carried over from the manufacturing process or added externally.

In a particular embodiment, the aqueous liquid composition comprises or consists essentially of 4 mg/mL olipudase alfa, 20 mM sodium phosphate, 100 mM methionine, and 5% (w/v) sucrose and has a pH of 6.5. The term “consists essentially of” means that the composition does not contain other ingredients at detectable amounts or may contain only trace amounts of certain materials that are derived from the protein manufacturing process where such materials do not affect the biological activity of the enzyme or causes harm in human patients.

In some embodiments, the composition is an aqueous liquid composition comprising 1-20 mg/mL (e.g., 10 mg/mL) rhASM (e.g., olipudase alfa) and 10-50 mM (e.g., 20 mM) sodium phosphate. In certain embodiments, the aqueous liquid composition further comprises methionine (e.g., L-methionine) and sucrose or trehalose. In certain embodiments, the aqueous liquid composition further comprises 80-120 mM (e.g., 100 mM) methionine and 4-6% (e.g., 5%) (w/v) sucrose. In particular embodiments, the aqueous liquid composition has a pH of 6.5.

In some embodiments, the composition is an aqueous liquid composition comprising 1-50 mg/mL (e.g. 3.8, 18, or 49 mg/mL) rhASM (e.g., olipudase alfa) and 10-50 mM (e.g., 20 mM) sodium phosphate. In certain embodiments, the aqueous liquid composition further comprises 1-15% (e.g. 5%, 6%, 7%, or 8%) sucrose or trehalose. In certain embodiments, the aqueous liquid composition further comprises 80-120 mM (e.g., 100 mM) methionine. In particular embodiments, the aqueous liquid composition has a pH of 6.5. The composition may comprise, for example, 3.8 mg/mL rhASM, 20 mM sodium phosphate, and 5% sucrose; 18 mg/mL rhASM, 20 mM sodium phosphate, and 5% sucrose; or 49 mg/mL rhASM, 20 mM phosphate, and 8% sucrose.

The aqueous liquid compositions may be prepared by mixing a human ASM produced by recombinant technology and subsequently purified from host cells with excipients described herein in water, and adjusting the resulting mixture to the desired pH. For example, the human ASM and desired excipients may be added to, or buffer-exchanged into, a sodium phosphate buffer with the desired sodium phosphate concentration and pH.

In some embodiments, the aqueous liquid composition may be prepared by reconstituting a lyophilized composition of the invention further described in detail below. The reconstitution may be done with a pharmaceutically acceptable liquid such as sterile water, saline (e.g., 0.9% sodium chloride), or phosphate-buffered saline.

Lyophilized Compositions

The present invention also provides lyophilized compositions. Such compositions can be prepared by lyophilizing the aqueous liquid compositions described herein. Lyophilized compositions are suitable for long term storage. Lyophilization may be performed according to methods known in the art. For example, a liquid composition may be cooled to a subzero (Celsius) temperature (e.g., −5° C. to −80° C.) that allows freezing, and then placed in a low pressure (partial vacuum) chamber to allow sublimation to occur (primary drying); where desired, the temperature of the composition may be raised in a second stage of drying (secondary drying) to further remove unwanted water molecules. In some embodiments, after completion of the lyophilization process, an inert gas such as nitrogen may be introduced into the container of the composition (e.g., a glass vial) before the container is sealed.

In some embodiments, the present invention provides powdered compositions, which may be prepared, e.g., by spray-drying the aqueous liquid compositions described herein. Spray-dried compositions are suitable for long term storage. Spray-drying may be performed according to methods known in the art. For example, a liquid composition may be forced through an atomizer or spray nozzle to disperse it as controlled-size tiny droplets into a hot gas stream in a chamber, resulting in rapid drying of the liquid composition to powder. The dried powder may then be collected at the bottom of the drying chamber. Other drying methods for preparing powdered compositions are also contemplated.

The inventors have unexpectedly discovered that sucrose (or trehalose) and methionine present at amounts described herein provide superior results during lyophilization; the lyophilized products form elegant cakes while preserving the stability of the human ASM during storage. The human ASM in the lyophilized compositions of the present invention may remain free of aggregation and biologically active for at least 4 months (e.g., at least 6 months or at least 12 months) under refrigerated conditions (e.g., at 0-10° C., 2-8° C., or 4° C.).

In some embodiments, the composition of the invention is a lyophilized pharmaceutical composition comprising 4-50% olipudase alfa, 3-7% sodium phosphate, and 45-90% sucrose (all w/w percentages). In certain embodiments, the lyophilized composition comprises 5.5% olipudase alfa, 20.6% L-methionine, 2.3% sodium phosphate dibasic heptahydrate, 2.6% sodium phosphate monobasic monohydrate, and 69.0% sucrose (all w/w percentages). In certain embodiments, the lyophilized composition comprises 6.6% olipudase alfa, 3.0% sodium phosphate dibasic heptahydrate, 3.3% sodium phosphate monobasic monohydrate, and 87.1% sucrose (all w/w percentages). In certain embodiments, the lyophilized composition comprises 25.2% olipudase alfa, 2.4% sodium phosphate dibasic heptahydrate, 2.6% sodium phosphate monobasic monohydrate, and 69.9% sucrose (all w/w percentages). In certain embodiments, the lyophilized composition comprises 47.8% olipudase alfa, 1.7% sodium phosphate dibasic heptahydrate, 1.8% sodium phosphate monobasic monohydrate, and 48.8% sucrose (all w/w percentages).

In some embodiments, the composition of the invention is a lyophilized pharmaceutical composition comprising 4-7% olipudase alfa, 15-25% L-methionine, 3-7% sodium phosphate, and 65-75% sucrose (all w/w percentages). In a particular embodiment, the lyophilized composition comprises 5.5% olipudase alfa, 20.5% L-methionine, 2.3% sodium phosphate dibasic heptahydrate, 2.6% sodium phosphate monobasic monohydrate, and 68.6% sucrose (all w/w percentages). In certain embodiments, the lyophilized composition may also comprise, e.g., 0.1%, 0.2%, 0.3%, 0.4%, 0.5%, 0.6%, 0.7%, 0.8%, 0.9%, or 1.0% moisture.

In some embodiments, the invention provides a vial containing a lyophilized pharmaceutical composition comprising 15-25 mg olipudase alfa, 75-85 mg L-methionine, 15-25 mg sodium phosphate, and 250-300 mg sucrose. Prior to use, the composition may be reconstituted in 4-6 mL of sterile water.

In some embodiments, the vial contains a lyophilized pharmaceutical composition comprising or consisting of 21.2 mg, 20.1 mg, 95.4 mg, or 259.7 mg of olipudase alfa; 9.0 mg sodium phosphate dibasic heptahydrate; 10.0 mg sodium phosphate monobasic monohydrate; and 265 mg sucrose. The lyophilized composition may optionally comprise 79.1 mg L-methionine. The lyophilized pharmaceutical composition may optionally comprise 0-0.3 mg (e.g., 0.08-0.16 mg) zinc, which may be, e.g., carried over from the manufacturing process or added externally. In certain embodiments, the vial may have an internally sterile nitrogen filled atmosphere. In a particular embodiment, the lyophilized composition may be reconstituted in 5.1 mL of sterile water to yield an olipudase alfa concentration of about 4.0 mg/mL, 3.8 mg/mL, 18 mg/mL, or 49 mg/mL, respectively. The reconstituted composition may be further diluted in 0.9% sodium chloride solution to a specific volume based on the dose to be administered.

In a particular embodiment, the vial contains a lyophilized pharmaceutical composition comprising or consisting of 21.2 mg olipudase alfa, 79 mg L-methionine, 9.0 mg sodium phosphate dibasic heptahydrate, 10.0 mg sodium phosphate monobasic monohydrate, and 265 mg sucrose. The lyophilized pharmaceutical composition may optionally comprise 0-0.3 mg (e.g., 0.08-0.16 mg) zinc, which may be, e.g., carried over from the manufacturing process or added externally. In certain embodiments, the lyophilized pharmaceutical composition is in the form of a cake or a lyophilized powder. In certain embodiments, the vial may have an internally sterile nitrogen filled atmosphere. In a particular embodiment, the lyophilized composition may be reconstituted in 5.1 mL of sterile water to yield an olipudase alfa concentration of about 4.0 mg/mL. The reconstituted composition may be further diluted in 0.9% sodium chloride solution to a specific volume based on the dose to be administered.

In some embodiments, the invention provides a vial containing a lyophilized pharmaceutical composition comprising 3-5 mg olipudase alfa, 15-17 mg L-methionine, 3-5 mg sodium phosphate, and 50-60 mg sucrose. Prior to use, the composition may be reconstituted in 0.8-1.2 mL of sterile water.

In a particular embodiment, the vial contains a lyophilized pharmaceutical composition comprising or consisting of 4.8 mg olipudase alfa, 17.9 mg L-methionine, 2.0 mg sodium phosphate dibasic heptahydrate, 2.3 mg sodium phosphate monobasic monohydrate, and 60 mg sucrose. In certain embodiments, the lyophilized pharmaceutical composition is in the form of a cake or a lyophilized powder. The lyophilized composition may optionally comprise 0-0.06 mg zinc, which may be, e.g., carried over from the manufacturing process or added externally. In certain embodiments, the vial may have an internally sterile nitrogen filled atmosphere. In a particular embodiment, the lyophilized composition may be reconstituted in 1.1 mL of sterile water to yield an olipudase alfa concentration of about 4.0 mg/mL. The reconstituted composition may be further diluted in 0.9% sodium chloride solution to a specific volume based on the dose to be administered.

Articles of Manufacture

The compositions of the invention may be supplied in an article of manufacture (e.g., a kit) that includes instructions for use and optionally other therapeutic agents for treating ASM disorders. The pharmaceutically active ingredient in the articles (e.g., the rhASM) may be supplied in an amount that can be readily administered in accordance to the dosing regimens described herein. For example, a “starter kit” may include multiple vials of varying amounts of rhASM for use in a dose escalation regimen.

For example, the article of manufacture may include a vial that contains 15-25 mg olipudase alfa, 75-85 mg L-methionine, 15-25 mg sodium phosphate, and 250-300 mg sucrose. In a particular embodiment, the article provides a lyophilized composition comprising 21.2 mg olipudase alfa, 79 mg methionine, 9.0 mg sodium phosphate dibasic heptahydrate, 10.0 mg sodium phosphate monobasic monohydrate, and 265 mg sucrose.

By way of another example, the article of manufacture may include a vial that contains 3-5 mg olipudase alfa, 15-17 mg L-methionine, 3-5 mg sodium phosphate, and 50-60 mg sucrose. In a particular embodiment, the article provides a lyophilized composition comprising 4.8 mg olipudase alfa, 17.9 mg L-methionine, 2.0 mg sodium phosphate dibasic heptahydrate, 2.3 mg sodium phosphate monobasic monohydrate, and 60 mg sucrose.

In some embodiments, the article of manufacture may further include a solution (e.g. sterile water, 0.9% sodium chloride, and/or phosphate-buffered saline) for reconstituting the lyophilized composition and/or further diluting the reconstituted composition prior to administration to a patient.

### Use of Acid Sphingomyelinase Compositions

The pharmaceutical compositions of the invention may be administered parenterally to a patient in need thereof as enzyme replace therapy. “Parenteral administration” refers to means of administration other than enteral and topical administration, usually by injection. Parenteral administration includes, without limitation, intravenous infusion or injection, and intramuscular, intradermal, intraperitoneal, and subcutaneous injection. In a particular embodiment, the pharmaceutical composition is administered via intravenous infusion.

The appropriate dosage level of the pharmaceutical composition described herein may be determined on the basis of a variety of factors, including the patient's age, weight, disease condition, general health, and medical history, as well as the route and frequency of the drug administration, the pharmacodynamics and pharmacokinetics of the ASM active ingredient in the drug, and any other drugs that the patient may be taking concurrently. In some embodiments, a pharmaceutical composition described herein may be administered according to a dosage regimen described in, e.g., U.S. Pat. No. 9,655,954 (Schuchman et al.). For example, the patient may receive escalating doses of the human ASM, with the dose strength starting at, e.g., 0.1 mg/kg or lower, and ending at 3 mg/kg (maintenance dose) or lower, depending on the patient's age and condition. In some embodiments, the first one or two doses may be given at a dose strength of 0.03 mg/kg or 0.1 mg/kg for a pediatric patient, or 0.1 mg/kg for an adult patient; after the patient has received one or two doses at 0.03 and/or 0.1 mg/kg, the patient is then given subsequent, sequential doses of 0.3 mg/kg, 0.3 mg/kg, 0.6 mg/kg, 0.6 mg/kg, 1.0 mg/kg, 2.0 mg/kg, and 3.0 mg/kg. In certain embodiments, any of said doses may be repeated (e.g., the doses at 1.0 mg/kg and 2.0 mg/kg). For some patients, the 3.0 mg/kg dose strength is suitable for the maintenance doses, while for other patients, a lower dose strength may be sufficient for maintenance. Intervals between successive doses may be two weeks, or shorter or longer than two weeks as determined to be appropriate by a clinician.

The invention provides a method of using a pharmaceutical composition described herein to treat ASMD in a patient in need thereof, a pharmaceutical composition described herein for use in treating ASMD in a patient in need thereof, and the use of a pharmaceutical composition described herein for the manufacture of a medicament for treating ASMD in a patient in need thereof. In some embodiments, the pharmaceutical composition may be a lyophilized composition, which may be reconstituted in a pharmaceutically acceptable liquid, such as sterile water, 0.9% sodium chloride solution, or phosphate-buffered saline.

The patients may be adults (e.g., patients 18 years or older, including geriatric patients who are 65 years or older). The patients may be pediatric patients (patients who are younger than 18 years old, e.g., patients who are newborn to 6 years old, who are 6 to 12 years old, or who are 12 to 18 years old). In some embodiments, the patients may have NPD AB or NPD B. In some embodiments, the patients may have NPD A. In particular embodiments, the pharmaceutical composition is for treating an adult or pediatric patient with chronic visceral ASMD (NPD B). In particular embodiments, the pharmaceutical composition is for treating non-neurological manifestations of ASMD in an adult or pediatric patient.

### Exemplary Embodiments

Further particular embodiments of the present invention are described as follows.

1. A composition comprising a recombinant human acid sphingomyelinase, sodium phosphate, methionine, and sucrose.
2. The composition of embodiment 1, wherein the composition is a lyophilized composition comprising:

4-7% w/w olipudase alfa (SEQ ID NO:2),

3-7% w/w sodium phosphate,

15-25% w/w L-methionine, and

65-75% w/w sucrose.

3. The composition of embodiment 2, consisting essentially of:

5.5% w/w olipudase alfa,

2.3% w/w sodium phosphate dibasic heptahydrate,

2.6% w/w sodium phosphate monobasic monohydrate,

20.5% w/w L-methionine, and

68.6% w/w sucrose.

4. The composition of embodiment 1, wherein the composition is an aqueous liquid composition comprising:

1-10 mg/mL olipudase alfa,

10-50 mM sodium phosphate,

70-150 mM L-methionine, and

1-10% w/v sucrose,

wherein the composition has a pH of 5-8.
5. The composition of embodiment 4, wherein the composition is an aqueous liquid composition comprising:

3-5 mg/mL olipudase alfa,

10-30 mM sodium phosphate,

80-120 mM L-methionine, and

4-6% w/v sucrose,

wherein the composition has a pH of 6-7.
6. The composition of embodiment 4, consisting essentially of:

4 mg/mL olipudase alfa,

20 mM sodium phosphate,

100 mM L-methionine, and

5% w/v sucrose,

wherein the composition has a pH of 6.5.
7. The composition of any one of embodiments 4-6, further comprising 0.005% w/v polysorbate 80.
8. A composition obtained by lyophilizing the aqueous liquid composition of any one of embodiments 4-7.
9. A process for manufacturing a lyophilized composition, comprising:

obtaining the aqueous liquid composition of any one of embodiments 4-7, and

lyophilizing the aqueous liquid composition.

10. A vial containing a lyophilized composition consisting essentially of:

21.2 mg olipudase alfa,

9.0 mg sodium phosphate dibasic heptahydrate,

10.0 mg sodium phosphate monobasic monohydrate,

79 mg L-methionine, and

265 mg sucrose.

11. An aqueous liquid composition obtained by reconstituting a lyophilized composition consisting essentially of:

21.2 mg olipudase alfa,

9.0 mg sodium phosphate dibasic heptahydrate,

10.0 mg sodium phosphate monobasic monohydrate,

79 mg L-methionine, and

265 mg sucrose,

in 5.1 mL of sterile water.
12. A vial containing a lyophilized composition consisting essentially of:

4.8 mg olipudase alfa,

2.0 mg sodium phosphate dibasic heptahydrate,

2.3 mg sodium phosphate monobasic monohydrate,

17.9 mg L-methionine, and

60 mg sucrose.

13. An aqueous liquid composition obtained by reconstituting a lyophilized composition consisting essentially of:

4.8 mg olipudase alfa,

2.0 mg sodium phosphate dibasic heptahydrate,

2.3 mg sodium phosphate monobasic monohydrate,

17.9 mg L-methionine, and

60 mg sucrose

in 1.1 mL of sterile water.
14. An article of manufacture comprising the vial of embodiment 10 or 12 and a vial containing sterile water, 0.9% sodium chloride, or phosphate-buffered saline for reconstituting the lyophilized composition.
15. A method of treating acid sphingomyelinase deficiency (ASMD) in a human patient, comprising administering to the patient the composition of any one of embodiments 1-8, 11, and 13, wherein the composition is reconstituted into a liquid form prior to administration if it is a lyophilized composition.
16. A composition of any one of embodiments 1-8, 11, and 13 for use in treating ASMD in a human patient.
17. Use of a composition of any one of embodiments 1-8, 11, and 13 for the manufacture of a medicament for treating ASMD in a human patient.
18. The method of embodiment 15, the composition for use of embodiment 16, or the use of embodiment 17, wherein the ASMD is Niemann-Pick Disease type AB or type B.
19. The method, composition for use, or use of embodiment 18, wherein the treatment is for non-neurological manifestations of ASMD.

All publications and other references mentioned herein are incorporated by reference in their entirety. Although a number of documents are cited herein, this citation does not constitute an admission that any of these documents forms part of the common general knowledge in the art. Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, microbiology, genetics, analytical chemistry, synthetic organic chemistry, medicinal and pharmaceutical chemistry, and protein and nucleic acid chemistry described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

In order that this invention may be better understood, the following examples are set forth. These examples are for purposes of illustration only and are not to be construed as limiting the scope of the invention in any manner.

### EXAMPLES

### Example 1: Recombinant Human Acid Sphingomyelinase Formulation

This Example describes studies that assessed the stability of various olipudase alfa aqueous liquid and lyophilized compositions.

### Materials and Methods

Solution Turbidity

Solution opalescence was assessed by a spectroscopic turbidity assay. Optical density in the 340-360 nm range was used to set ranges for previously established categories of opalescence based on European Pharmacopoeia reference suspensions at specific NTU values. Analysis was performed on a SpectraMax Plus 384 Microplate Spectrophotometer (Molecular Devices, Sunnyvale, Calif.).

Aggregation

Aggregation and dimer analysis was performed by SEC. Prior to loading into HPLC vials, each sample was mixed by five gentle pipette cycles. SEC analysis was performed on a 1100/1200 series HPLC (Agilent, Santa Clara, Calif.) equipped with a TSK gel G3000SWXL (Tosoh Bioscience, Tokyo, Japan) analytical column and matching guard column. The mobile phase used was 20 mM sodium phosphate at pH 6, 200 mM sodium chloride set at a flow rate of 0.5 mL min′ for 35 minutes. Three injections of approximately 80 μg were performed for each sample. Detection was carried out by UV absorbance at 280 nm.

The level of olipudase alfa related high molecular weight species (HMWS) was determined using SDS-PAGE under non-reducing conditions followed by staining with Coomassie Blue. The olipudase alfa reference standard is included on each gel. Olipudase alfa samples were mixed with sample buffer and loaded onto a 4-20% Tris-Glycine gradient gel along with molecular weight markers. Following electrophoresis at a target of 125 V for approximately 2 hours, the gels were stained with Coomassie Blue and de-stained in methanol, acetic acid and HPLC grade water. Densitometric analysis was performed to provide quantitative results on the percentage of the HMWS bands relative to all observed bands.

Enzyme Activity

rhASM samples were diluted 2000:1 in 1.2 mL library tubes. The rate of hydrolysis of 2-(N-hexadecanoylamino)-4-nitrophenylphosphorylcholine (HDA-PC) at 37° C. catalyzed by rhASM was measured in this procedure. The released chromophore was measured by absorbance at 415 nm using a SpectraMax Plus 384 Microplate Spectrophotometer. One unit of rhASM activity is defined as the amount of enzyme that generates one μmol of 2-(N-hexadecanoylamino)-4-nitrophenol per minute from HDA-PC under the specified assay conditions.

Protein Concentration

The protein concentration of the rhASM samples was determined by absorbance at 280 nm. Samples were diluted in duplicate to 1:10 and 1:20 using matching buffer. Absorbance at 280 nm was determined on a SpectraMax Plus 384 Microplate Spectrophotometer.

pH

Sample pH analysis was performed on a Thermo Electron Microprobe pH meter (Thermo Scientific, Beverly, Mass.). A Thermo Orion 8203BN PerHecT Ross Semi micro glass probe (Thermo Scientific) was used.

Differential Scanning Calorimeter

Differential scanning calorimeter (DSC) analysis was performed using a CAP-VP-DSC microcalorimeter (MicroCal-GE Healthcare, Northampton, Mass.). Samples were diluted to 0.4 mg/mL with the matching buffer. Samples were run at a scan rate of 200° C./hr from 15-100° C. Data analysis was performed in Origin 7.0 (OriginLab Corp., Northampton, Mass.) equipped with the DSC analysis add-on (MicroCal-GE Healthcare).

### Results

Buffer and pH Evaluation

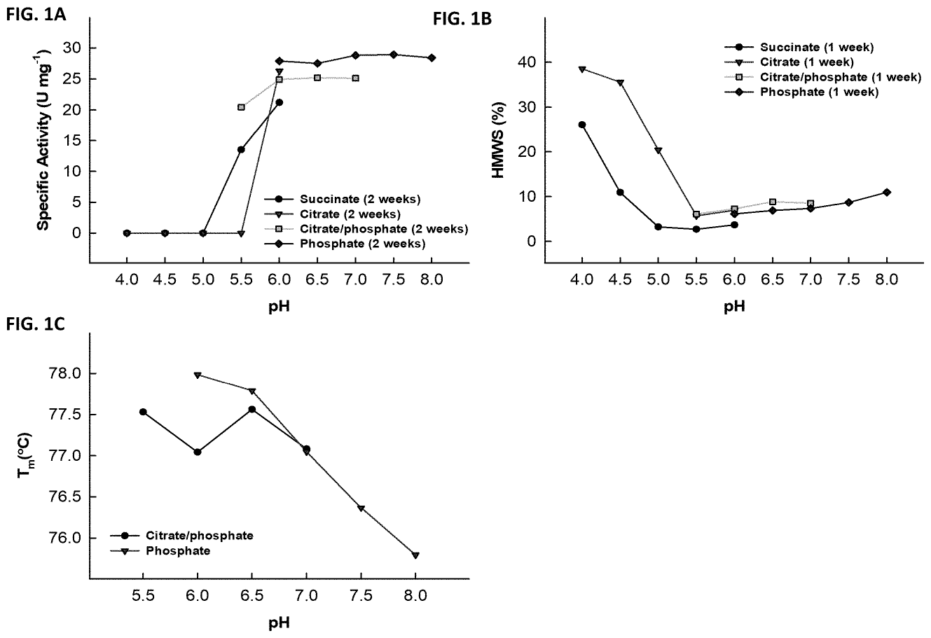

A variety of buffer pH and buffer species were evaluated for their effects on rhASM stability. The assays were conducted by incubating 4 mg/ml olipudase alfa in 20 mM buffer at 30° C. for two weeks and assessing physical and function stability of the enzyme (FIGS. 1A-1C).

Significant instability, both physical and functional, was observed in olipudase alfa below pH 6.0. Enzymatic activity decreased precipitously below pH 6.0 but remained relatively constant at pH 6.0 and above (FIG. 1A). Aggregation propensity was at a minimum between pH 5.5 and 6.5. Rapid increases in aggregation were observed below pH 5.5 and gradual increases in aggregation were seen above pH 6.5 (FIG. 1B). At comparable pH values, stability was higher in phosphate buffer compared to citrate/phosphate buffer. These stability trends were consistent with data obtained from samples stored at refrigeration temperatures. The gradually increasing aggregation rates observed above pH 6.5 were corroborated by DSC analysis, which showed decreased thermal stability at higher pH in phosphate buffer (FIG. 1C). Based on these data, sodium phosphate buffer with a pH of about 6.5 was identified as a suitable buffering system for the rhASM formulation.

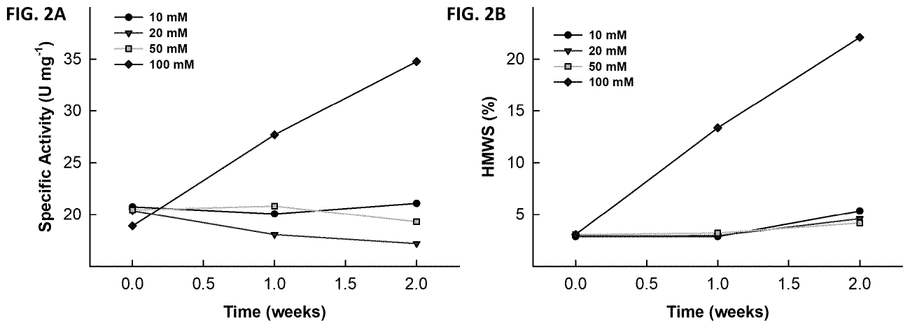

Next, the impact of ionic strength, or buffer concentration, on stability was investigated by varying the sodium phosphate concentration from 10 mM to 100 mM. As shown in FIGS. 2A and 2B, ionic strength at low buffer concentrations (10 mM, 20 mM, or 50 mM) had little effect on the enzymatic activity and physical stability of olipudase alfa. The stability of olipudase alfa in sodium phosphate, pH 6.5 was relatively consistent for buffer concentrations of 50 mM and below. However, there was a notable decrease in stability when the concentration of sodium phosphate was increased to 100 mM. Such a concentration resulted in a rapid increase in aggregate species and concomitant reduction in activity (FIGS. 2A and 2B). Data at 30° C. were consistent with those seen during storage of olipudase alfa under refrigerated conditions. A sodium phosphate concentration of 20 mM was selected as a buffer concentration that while low, still ensured sufficient buffering capacity.

Excipient Evaluation

A variety of pharmaceutically acceptable excipients were evaluated for their impact on the stability of olipudase alfa in liquid and lyophilized compositions. The impact of known stabilizers acting through a preferential exclusion mechanism on liquid stability was investigated first (see, e.g., Timasheff, Proc Nat Acad Sci USA. 99: 9721-9726 (2002); and Lee et al., J. Biol. Chem. 256:7193-7201 (1981)). Sucrose, trehalose and propylene glycol were added to the base formulation of 20 mM sodium phosphate, 0.005% PS80, pH 6.5 and assessed for their impact on the enzymatic activity and physical stability of 4 mg/mL olipudase alfa. The data showed that the presence of any of these preferential exclusion stabilizers did not enhance the stability of olipudase alfa in liquid state at 5° C. or 30° C. (data not shown). While there was a loss of enzymatic activity and physical stability under all conditions studied, sucrose appeared slightly more favorable than trehalose and propylene glycol.

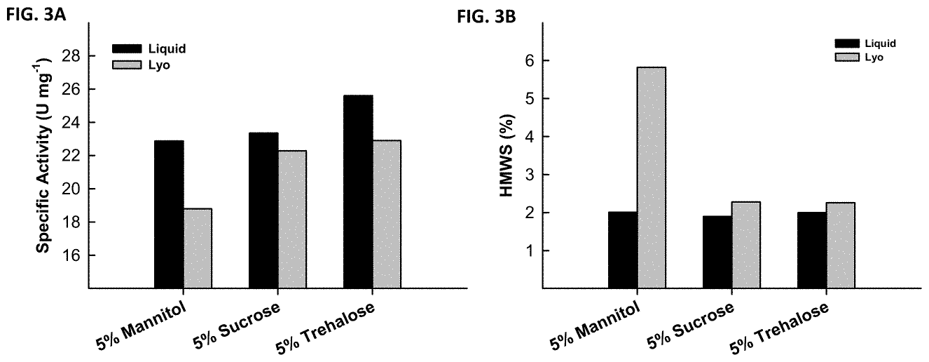

Next, the impact of sucrose and trehalose on the stability of olipudase alfa during freeze-drying was investigated. Mannitol was also investigated as it is a commonly used bulking agent in freeze-drying (FIGS. 3A and 3B). The three polyols were included at 5% w/v in the liquid compositions, which were subsequently lyophilized. The data in FIG. 3A show that the addition of mannitol decreased the enzymatic activity of olipudase alfa after freeze-drying. The data in FIG. 3B show that the addition of mannitol resulted in a large increase in protein aggregation after freeze-drying. Increases in aggregation in the presence of sucrose and trehalose were minimal. Sucrose was chosen as a cryoprotectant at a concentration of 5% w/v in liquid compositions, which could then be lyophilized.

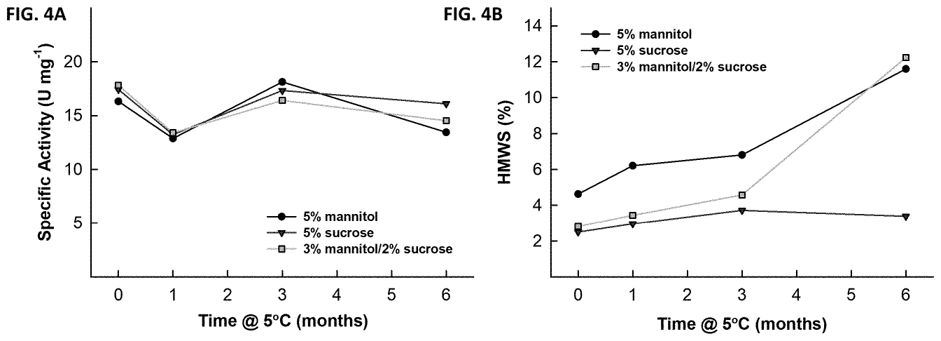

The stability of lyophilized olipudase alfa formulations over time in the presence of mannitol was also investigated. Olipudase alfa was freeze-dried from liquid compositions containing 20 mM sodium phosphate buffer (pH 6.5), 0.005% PS80 and (i) 5% w/v mannitol, (ii) 5% w/v sucrose, or (iii) 3% mannitol and 2% sucrose). The data show that mannitol not only resulted in immediate as well as continuous increases in protein aggregation during freeze-drying when used alone, but it also did so when used in combination with sucrose during a six month period (FIGS. 3B and 4B). Enzymatic activity at six months was also lower in the presence of mannitol than in the absence of mannitol (FIG. 4B). Accordingly, mannitol was deemed an unsuitable bulking agent for lyophilized rhASM formulations.

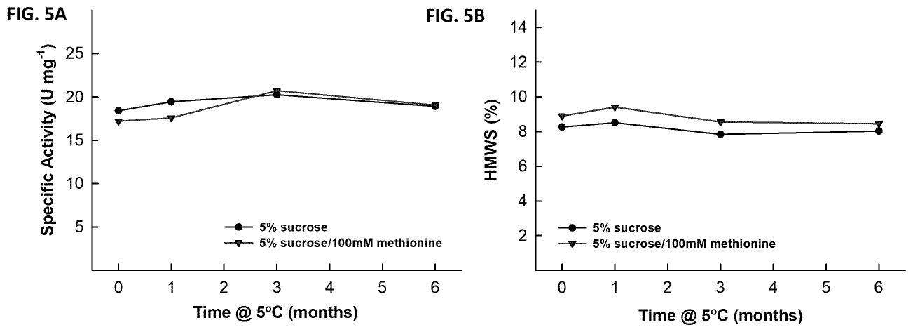

As mannitol proved to be deleterious for rhASM stability during freeze-drying, methionine was evaluated as a potential bulking agent. Enzymatic activity and physical stability during liquid storage of olipudase alfa in the presence of L-methionine was assessed, and it was found that the addition of 100 mM L-methionine neither improved nor negatively impacted the liquid stability of olipudase alfa at 5° C. As shown in FIGS. 5A and 5B, lyophilized compositions prepared from liquid compositions containing 100 mM L-methionine resulted in a stable lyophilized configuration for olipudase alfa. No changes in activity or aggregation were observed over the course of six months of storage at 5° C. Additionally, cakes obtained from freeze-drying olipudase alfa in the presence of methionine and sucrose had an improved appearance over those lyophilized with just sucrose.

Identification of an appropriate amount of methionine for lyophilization was performed by X-ray diffraction (XRD) of olipudase alfa cakes lyophilized from liquid compositions containing 20 mM sodium phosphate (pH 6.5), 5% w/v sucrose, and increasing amounts of L-methionine (data not shown). A sample containing no methionine was completely amorphous. There was some evidence of crystallinity in samples containing 33 mM methionine, and more in samples containing 66 mM and 100 mM methionine. Lyophilization of olipudase alfa at methionine levels below 100 mM (e.g., 10 mM and 33 mM) resulted in vials that appeared dimpled and collapsed (shrunken). Hence, 100 mM methionine was chosen as a bulking agent for lyophilized rhASM formulations.

### Example 2: Robustness of Olipudase Alfa Compositions

To assess the robustness of compositions containing olipudase alfa, sucrose, and L-methionine, each of these components was set at five different levels relative to the control—low, medium low, center (control), medium high, and high—in a 20 mM sodium phosphate buffer (Table 1).

TABLE 1
Excipient Levels for Evaluating Olipudase Alfa Formulation Robustness

| | | | Med. | | Med. | |
|---|---|---|---|---|---|---|
| Components | Control | Low | Low | Center | High | High |
| Sucrose (% w/v) | 5 | 4 | 4.33 | 5 | 5.67 | 6 |
| L-Methionine (mM) | 100 | 80 | 86.51 | 100 | 113.49 | 120 |
| Olipudase (mg/mL) | 4 | 3 | 3.33 | 4 | 4.67 | 5 |
| pH | 6.5 | 6 | 6.16 | 6.5 | 6.84 | 7 |

A total of 26 liquid formulation variants were generated (Table 2). Formulations 2 and 7 represent control formulations, or center points. The remaining 24 formulation variants represent various conditions around the center points. All 26 variants were stored at 2-8° C. (24 weeks or up to 12 months) and 25° C. (16 weeks) to monitor their stability.

TABLE 2
Olipudase Alfa Formulations for Evaluation of Robustness

| | Sucrose | Methionine | Olipudase | |
|---|---|---|---|---|
| Formulation | (%) | (mM) | (mg/mL) | pH |
| 1 | 5.00 | 100.00 | 3.00 | 6.50 |
| 2 | 5.00 | 100.00 | 4.00 | 6.50 |
| 3 | 5.00 | 80.00 | 4.00 | 6.50 |
| 4 | 5.00 | 100.00 | 5.00 | 6.50 |
| 5 | 5.00 | 120.00 | 4.00 | 6.50 |
| 6 | 6.00 | 100.00 | 4.00 | 6.50 |
| 7 | 5.00 | 100.00 | 4.00 | 6.50 |
| 8 | 4.00 | 100.00 | 4.00 | 6.50 |
| 9 | 5.67 | 113.49 | 4.67 | 6.16 |
| 10 | 5.67 | 113.49 | 3.33 | 6.16 |
| 11 | 4.33 | 113.49 | 3.33 | 6.16 |
| 12 | 4.33 | 113.49 | 4.67 | 6.16 |
| 13 | 4.33 | 86.51 | 4.67 | 6.16 |
| 14 | 4.33 | 86.51 | 3.33 | 6.16 |
| 15 | 5.67 | 86.51 | 3.33 | 6.16 |
| 16 | 5.67 | 86.51 | 4.67 | 6.16 |
| 17 | 5.67 | 113.49 | 3.33 | 6.84 |
| 18 | 5.67 | 86.51 | 4.67 | 6.84 |
| 19 | 5.67 | 113.49 | 4.67 | 6.84 |
| 20 | 4.33 | 86.51 | 3.33 | 6.84 |
| 21 | 4.33 | 113.49 | 3.33 | 6.84 |
| 22 | 4.33 | 86.51 | 4.67 | 6.84 |
| 23 | 4.33 | 113.49 | 4.67 | 6.84 |
| 24 | 5.67 | 86.51 | 3.33 | 6.84 |
| 25 | 5.00 | 100.00 | 4.00 | 7.00 |
| 26 | 5.00 | 100.00 | 4.00 | 6.00 |

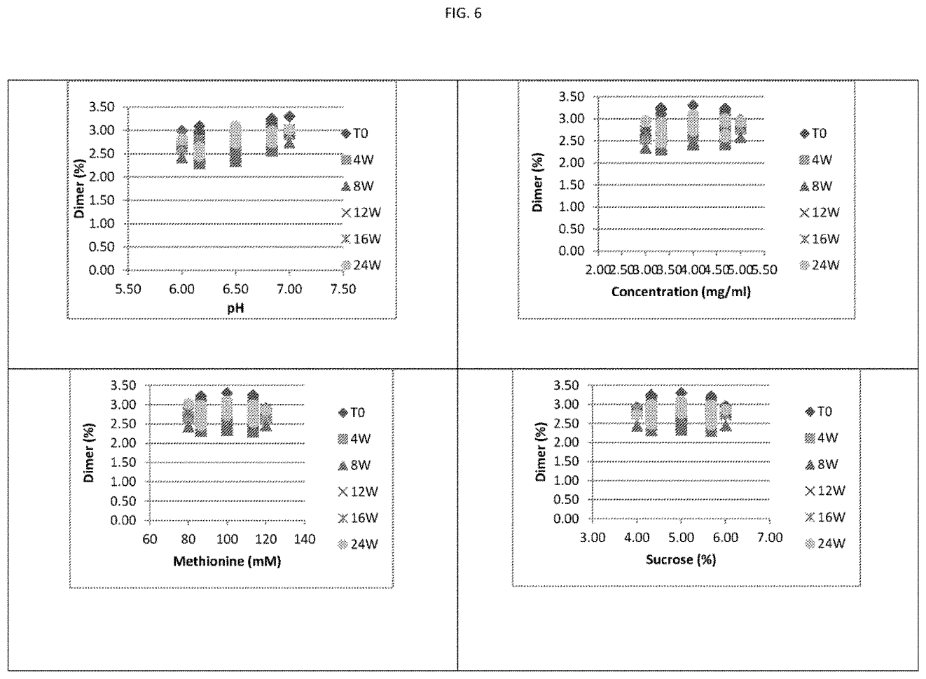

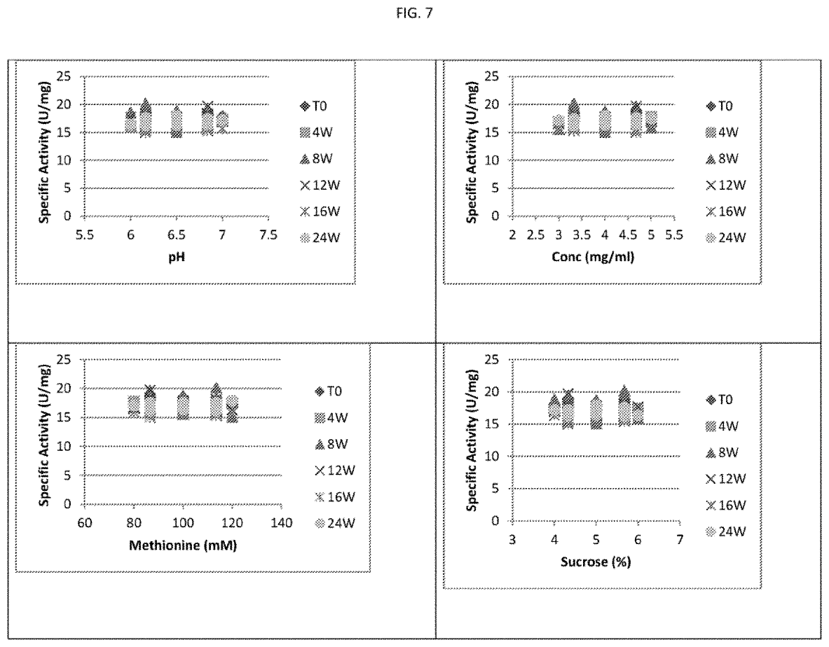

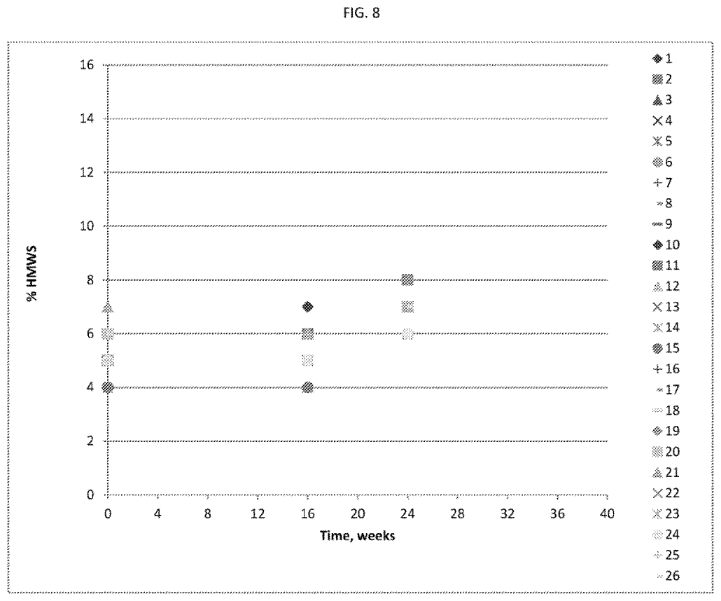

At the end of the test periods, the following parameters would indicate stability of the compositions: (1) clear, colorless appearance; (2) pH 6.0-7.0; (3) no more than 3.0% of aggregate; (4) 3.5-4.5 mg/mL protein; (5) no more than 15% of HMWS; and (6) specific activity of 11-42 U/mg. FIGS. 6-8 show the effects of different factors on % dimer, specific activity, and % HMWS, respectively, in the 26 formulation variants following 24 weeks of storage at 2-8° C. The data shows that there was no significant difference among the variants with respect to % dimer, specific activity, or % HMWS and thus that all 24 variants along with the two control formulations were stable at 2-8° C. for 24 weeks.

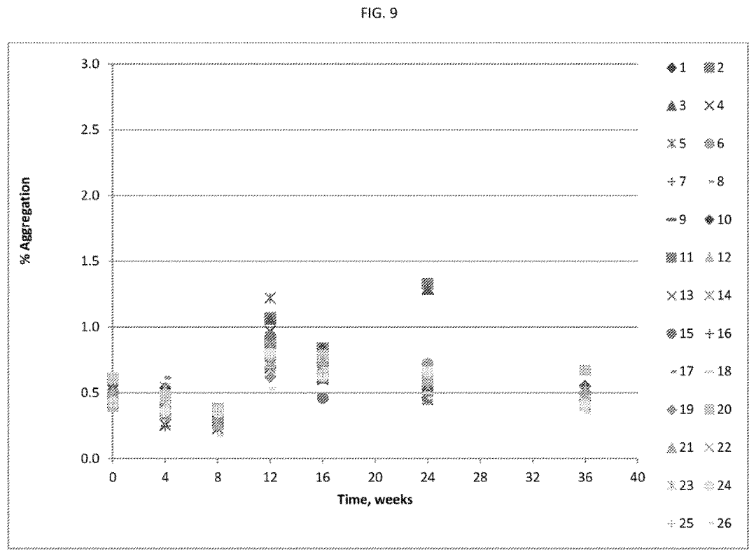

The lack of significant effects of the varying pH and ingredient concentrations on dimer and specific activity was also observed for some variants through 36 weeks. FIG. 9 shows that all of the variants have comparable aggregation at 36 weeks.

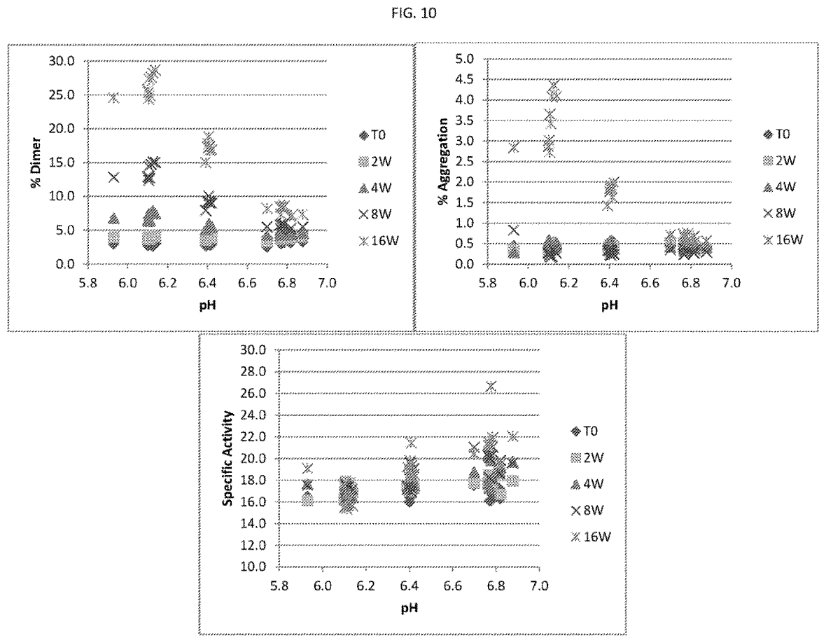

At an accelerated temperature of 25° C., there also was little effect of excipients on stability over 16 weeks in the ranges tested. The most prominent effect was observed in variants with different pH (FIG. 10). There was greater stability for olipudase alfa at a higher pH (6.8 to 7.0), and more aggregation and dimer at a lower pH (6.5 or lower).

This study shows that the variant formulations at 2-8° C. were robust and stable in the selected component ranges.

1. A composition comprising a recombinant human acid sphingomyelinase, sodium phosphate, methionine, and sucrose.

2. The composition of claim 1, wherein the composition is a lyophilized composition comprising:
4-7% w/w olipudase alfa (SEQ ID NO:2),
3-7% w/w sodium phosphate,
15-25% w/w L-methionine, and
65-75% w/w sucrose.

3. The composition of claim 2, consisting essentially of:
5.5% w/w olipudase alfa,
2.3% w/w sodium phosphate dibasic heptahydrate,
2.6% w/w sodium phosphate monobasic monohydrate,
20.5% w/w L-methionine, and
68.6% w/w sucrose.

4. The composition of claim 1, wherein the composition is an aqueous liquid composition comprising:
1-10 mg/mL olipudase alfa,
10-50 mM sodium phosphate,
70-150 mM L-methionine, and
1-10% w/v sucrose,
wherein the composition has a pH of 5-8.

5. The composition of claim 4, wherein the composition is an aqueous liquid composition comprising:
3-5 mg/mL olipudase alfa,
10-30 mM sodium phosphate,
80-120 mM L-methionine, and
4-6% w/v sucrose,
wherein the composition has a pH of 6-7.

6. The composition of claim 4, consisting essentially of:
4 mg/mL olipudase alfa,
20 mM sodium phosphate,
100 mM L-methionine, and
5% w/v sucrose,
wherein the composition has a pH of 6.5.

7. The composition of claim 1, further comprising 0.005% w/v polysorbate 80.

8. A composition obtained by lyophilizing the aqueous liquid composition of claim 4.

9. A process for manufacturing a lyophilized composition, comprising:
obtaining the aqueous liquid composition of claim 4, and
lyophilizing the aqueous liquid composition.

10. A vial containing a lyophilized composition consisting essentially of:
21.2 mg olipudase alfa,
9.0 mg sodium phosphate dibasic heptahydrate,
10.0 mg sodium phosphate monobasic monohydrate,
79 mg L-methionine, and
265 mg sucrose.

11. An aqueous liquid composition obtained by reconstituting a lyophilized composition consisting essentially of:
21.2 mg olipudase alfa,
9.0 mg sodium phosphate dibasic heptahydrate,
10.0 mg sodium phosphate monobasic monohydrate,
79 mg L-methionine, and
265 mg sucrose,
in 5.1 mL of sterile water.

12. A vial containing a lyophilized composition consisting essentially of:
4.8 mg olipudase alfa,
2.0 mg sodium phosphate dibasic heptahydrate,
2.3 mg sodium phosphate monobasic monohydrate,
17.9 mg L-methionine, and
60 mg sucrose.

13. An aqueous liquid composition obtained by reconstituting a lyophilized composition consisting essentially of:
4.8 mg olipudase alfa,
2.0 mg sodium phosphate dibasic heptahydrate,
2.3 mg sodium phosphate monobasic monohydrate,
17.9 mg L-methionine, and
60 mg sucrose
in 1.1 mL of sterile water.

14. An article of manufacture comprising the vial of claim 10 and a vial containing sterile water, 0.9% sodium chloride, or phosphate-buffered saline for reconstituting the lyophilized composition.

15. A method of treating acid sphingomyelinase deficiency (ASMD) in a human patient, comprising administering to the patient the composition of claim 1, wherein the composition is reconstituted into a liquid form prior to administration if it is a lyophilized composition

16-17. (canceled)

18. The method of claim 15, wherein the ASMD is Niemann-Pick Disease type A/B, or type B.

19. The method of claim 18, wherein the treatment is for non-neurological manifestations of ASMD.

20. The composition of claim 2, wherein the lyophilized composition comprises no more than 0.5% moisture.


SEQUENCE LISTING (6 sequences)

SEQ ID NO: 1 - protein, 627 aa, Homo sapiens
MARYGASLRQSCPRSGREQGQDGTAGAPGLLWMGLALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 2 - protein, 570 aa, Homo sapiens
HPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 3 - protein, 629 aa, Homo sapiens
MPRYGASLRQSCPRSGREQGQDGTAGAPGLLWMGLVLALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESIPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSRSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 4 - protein, 570 aa, Homo sapiens
HPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESIPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSRSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 5 - protein, 629 aa, Homo sapiens [sequence omitted]

SEQ ID NO: 6 - protein, 570 aa, Homo sapiens; identical to SEQ ID NO: 2